(19) United States
(12) Patent Application Publication
(10) Pub. No.: US 2006/0004258 A1
(43) Pub. Date: Jan. 5, 2006

(54) Image-type intubation-aiding device

(76) Inventors: Wei-Zen Sun, Taipei City (TW); Jung-Hsiang Hsu, Longtan Township (TW); Ping-Kuo Weng, Bade City (TW); Ker-Jer Huang, Longtan Township (TW); Zhi-Jun Zhan, Jhuolan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/882,200

(22) Filed: Jul. 2, 2004

(51) Int. Cl. A61B 1/06; A61B 1/04

(52) U.S. Cl. 600/160; 600/179; 600/131; 600/109

(57) ABSTRACT

An image-type intubation-aiding device comprises a small-size image sensor and a light source module both placed into an endotracheal tube to help doctors with quick intubation. Light from light emission devices in the light source module passes through a transparent housing and is reflected by a target and then focused. The optical signal is converted into a digital or analog electric signal by the image sensor for displaying on a display device after processing. Doctors can thus be helped to quickly find the position of trachea, keep an appropriate distance from a patient for reducing the possibility of infection, and lower the medical treatment cost. Disposable products are available to avoid the problem of infection. The intubation-aiding device can be used as an electronic surgical image examination instrument for penetration into a body. Moreover, a light source with tunable wavelengths can be used to increase the spot ratio of nidus.

### FIELD OF THE INVENTION

The present invention relates to an electronic surgical image examination instrument for penetration into a body and, more particularly, to an image-type intubation-aiding device for helping a doctor with the intubation of tracheal tube.

### BACKGROUND OF THE INVENTION

An endoscope is an instruments widely used in medicine. It is generally used to examine hollow internal organs or cavities. An endoscope can increase the brightness within the range of a wound and can also enlarge the field of vision for a doctor. A doctor can make use of an endoscope to perform an operation for many wounds without resulting in a larger wound.

Conventionally, many fibers bundled together with a charge couple device (CCD) used to take pictures to form an endoscope, which is used to penetrate hollow organs (e.g., stomach, large intestine and trachea) to get tissue images for determining the type and development degree of diseases. Light from a light source is transmitted through the fibers to illuminate a tissue of the human body. The reflected light is transmitted back via the fibers to the CCD for formation of an image displayed on a screen. The diameter of common fibers is smaller than 200 μm. In order to observe an image region from several millimeters to several centimeters, it is necessary to bundle a considerable number of fibers to obtain an image with a sufficient resolution. Moreover, the size of CCD is generally large. The above fiber-type endoscope has the disadvantages of high price and complexity and difficult assembly and maintenance. Because, the above fiber-type endoscope has a high price, it is usually used repetitively for many times so that infection may occur due to difficult sterilization.

In order to solve the above problems of the fiber-type endoscope, U.S. Pat. No. 6,387,043 discloses a transmission type endoscope, wherein a complementary metal-oxide semiconductor (CMOS) image sensor replaces the CCD. As shown in FIG. 1a, a transmission type endoscope 10 applies to common surgical operations or endoscopic operations. The transmission type endoscope 10 comprises a penetrating member 102, a hollow portal sleeve 104 connected with the penetrating member, and a main body 106 at the rear end. As shown in FIG. 1a, the penetrating member 102 has a sharp front end 1022 for penetrating tissues, LED light sources 1024 and 1026 for illumination, object lenses 1028 and 1030 for focusing images, and CMOS image sensors 1032 and 1034 for converting optical signals into electric signals. After the electric signals are sent to the main body 106 via signal lines 108 and 110 and then processed, images will be displayed on a display 112 disposed on the main body 106. A handle 114 for convenient holding is also disposed below the main body 106.

U.S. application Ser. No. 2002/0080248 A1 discloses an endoscope of another type. Light from the light source and reflected light are sent via fibers in conventional endoscopes. In this disclosure, the illumination way of the light source is reserved. Only the CCD image sensor is replaced with a CMOS image sensor. As shown in FIG. 2, an endoscope 20 comprises a flexible sleeve 202, a handle 204, and a control box 206. An optical imaging device 208 is installed at the front end of the flexible sleeve 202. The optical imaging device 208 comprises from outside to inside an outer cover 2082, fibers 2084, and an image sensing device 2086. An optical lens 210 is disposed at the front end of the image sensing device 2086. A CMOS sensor is disposed behind the image sensing device 2086. The CMOS sensor can be a circular image sensor 212 or a square image sensor 214. The handle 204 is used for convenient maneuvering of the endoscope 20. The control box 206 provides electric power and has an image processing board 216 for processing image signals.

Although the above two disclosures solve the problems of fiber-type endoscopes and avoid the situation of using too many fibers. The advantages of the CMOS image sensor like small size and power saving aren't fully made use of.

Accordingly, the present invention aims to propose an image-type intubation-aiding device to solve the above problems in the prior art.

### SUMMARY AND OBJECTS OF THE PRESENT INVENTION

The primary object of the present invention is to provide an image type intubation-aiding device comprising a small-size image sensor and a light source placed in an endotracheal tube to help doctors with quick intubation. Surgeon can rotate or move the probing device through the handle to quickly find the position of trachea. The image type intubation-aiding device of the present invention also applies to other hollow organs.

Another object of the present invention is to provide an image type intubation-aiding device, which makes use of the advantages of a CMOS image sensor like small size and power saving and new optical techniques to increase the spot ratio of nidus.

Another object of the present invention is to provide an image type intubation-aiding device, wherein a tiny CMOS image sensor and light emitting diodes (LED) or organic light emitting diodes (OLED) used as the illumination light source replace the conventional expensive and vulnerable fiber-type endoscope to effectively lower the cost of medical treatment.

Another object of the present invention is to provide an image type intubation-aiding device, whereby disposable endoscopes are available to avoid infection of the human body due to repetitive use of conventional endoscopes.

To achieve the above objects, the present invention proposes an image type intubation-aiding device comprising a probing device made of material compatible with the human body, a flexible soft tube, a display device, and a power source device. The probing device comprises a housing, a light source module behind the housing for illuminating the front, and an optical and imaging device behind the light source module for converting the optical signal into an electric signal. The flexible soft tube is connected with the probing device. The display device is connected with the flexible soft tube and the optical and imaging device. The display device is used to receive the electric signal for displaying after processing. The power source device is connected with all the above devices for providing electric power.

The various objects and advantages of the present invention will be more readily understood from the following detailed description when read in conjunction with the appended drawings, in which:

### BRIEF DESCRIPTION OF DRAWINGS

FIG. 1a is a perspective view of a conventional transmission type endoscope;

FIG. 1b is a perspective view of a penetrating member of a conventional transmission type endoscope;

FIG. 2 is a perspective view of a conventional endoscope;

FIG. 3a is a perspective view of the present invention;

FIG. 3b is an enlarged perspective view of a probing device of the present invention;

FIG. 4 is a rotation diagram of a display device of the present invention;

FIG. 5 is a structure diagram of a biopsy device in a flexible soft tube of the present invention;

FIG. 6 is a perspective view according to another embodiment of the present invention;

FIG. 7 is a diagram showing how an image is transmitted to a mask type head-up display of the present invention; and

FIG. 8 is a diagram showing how an image is transmitted to a handheld display of the present invention.

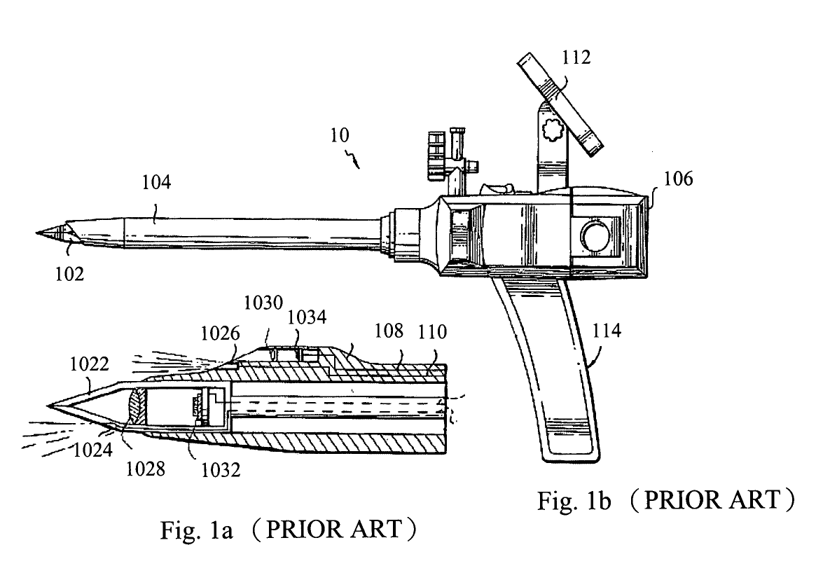

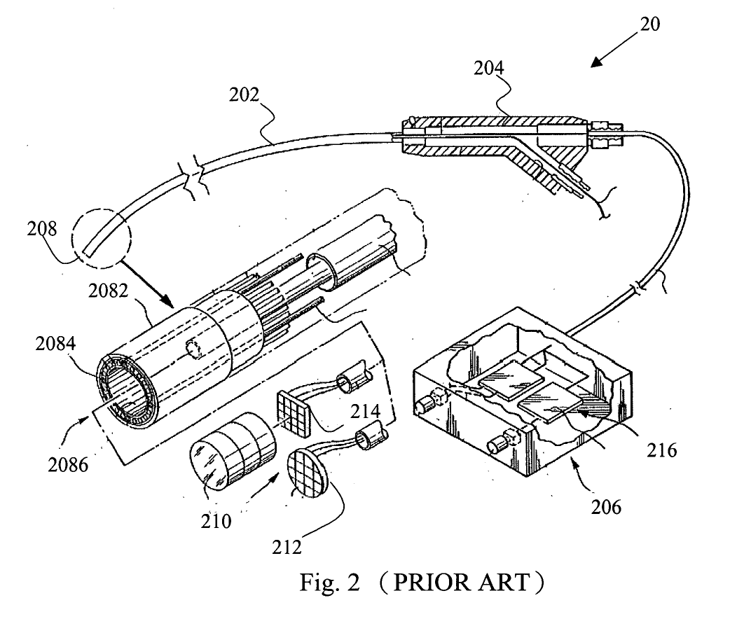

### DETAILED DESCRIPTION OF PREFERRED EMBODIMENT

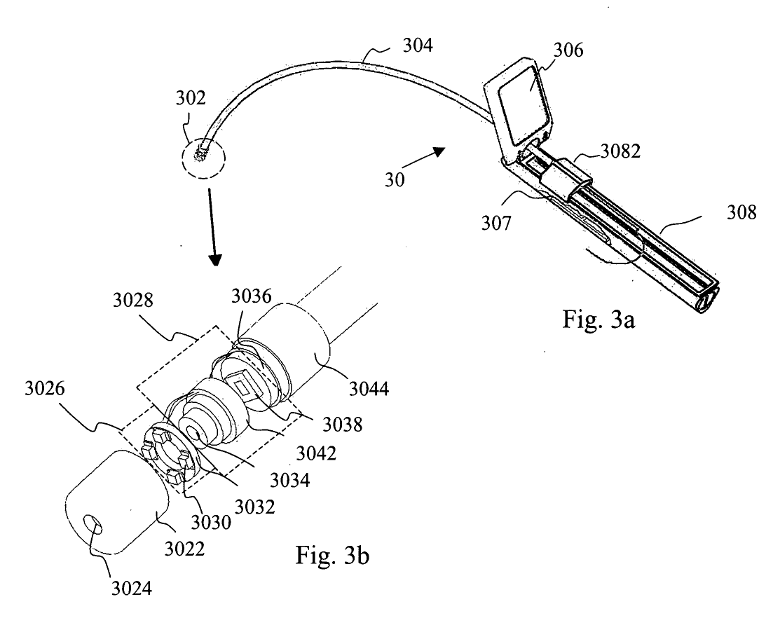

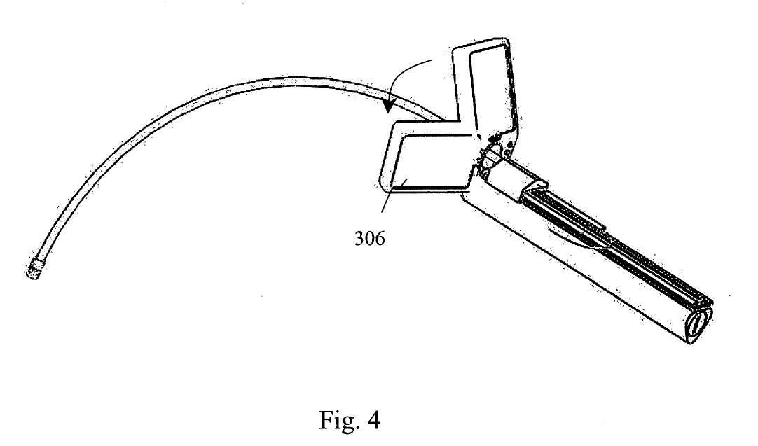

The present invention proposes an image type intubation-aiding device. As shown in FIG. 3a, an image type intubation-aiding device 30 comprises a probing device 302 made of material compatible with the human body. As shown in FIG. 3b, the probing device 302 comprises a housing 3022 with a diameter smaller than 15 mm. The housing 3022 is pervious to light or has several holes for light penetration. A light-collecting lens 3024 is disposed in the housing 3022. The light-collecting lens 3024 can be integrally formed with the housing 3022. The light-collecting lens 3024 is used for light collection to produce an optical signal. A light source module 3026 is disposed behind the housing 3022 for illuminating the front through the light-collecting lens 3024. An optical and imaging device 3028 is disposed behind the light source module 3026 for converting the optical signal into an electric signal like a digital signal or an analog signal. The image type intubation-aiding device 30 also comprises a flexible soft tube connected with the probing device 302. The image type intubation-aiding device 302 also comprises a black/white or color display device 306 capable of rotating for 360 degrees. The display device 306 can be a liquid crystal display (LCD), an organic light emitting display, or a cold cathode fluorescent lamp (CCFL). The display device 306 is connected with the flexible soft tube 304, and is connected to the optical and imaging device 3028 via electric wires. The display device 306 is rotatable to facilitate operation for medical staffs. The display device 306 receives the electric signal converted by the optical and imaging device 3028 for displaying after processing in a wired or wireless way. The image type intubation-aiding device 30 also comprises a power source device like a common AC power, a battery, or a rechargeable battery for providing electric power.

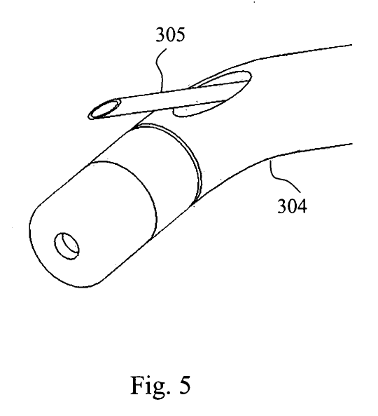

As shown in FIG. 5, a hole is formed on the flexible soft tube 304 with a biopsy device 305 disposed therein for sampling, sectioning, or inflation to facilitate sampling and providing oxygen for a patient in real time during intubation. Please refer to FIGS. 3a and 3b. There is a thick metal wire in the flexible soft tube 304. An operator holds the handle 308 to drive a soft tube retractable device 3082 for controlling the bend angle of the flexible soft tube 304. When the soft tube retractable device 3082 is pushed to the bottom, the thick metal wire penetrates deeply into the flexible soft tube 304 to straighten it; otherwise, the flexible soft tube 304 will bend. In order to the thick metal wire, there are also electric wires for transmission or electric power and signal in the flexible soft tube 304. The light source module 3026 comprises light emission devices 3030 of several wavelength bands like LEDs or OLEDs of white light, blue light, red light, other single color lights or mixed color lights. The housing 3022 is in front of the light emission devices 3030. Light from the light emission devices 3030 is transmitted through the light-collecting lens 3024 in the housing 3022 compatible with the human body and pervious to light to illuminate the front. The light source module 3026 also comprises a light source drive circuit 3032 for driving the light emission devices 3030 to emit light. The optical and imaging device 3028 comprises a focusing lens 3034 having a visual angle larger than 36 degrees, an image sensor 3038 (e.g., a CMOS or a CCD) disposed on an image sensor drive circuit board 3036 having a voltage-regulating capacitor. The focusing lens 3034 is fixed on a lens holder 3042. The image sensor 3038 converts the optical signal into an electric signal, and is sleeved in a cover body 3044 compatible with the human body. The power source device is disposed in the handle 308 behind and connected with the display device 306. A control circuit 307 is disposed in the handle 308 for capturing a video or taking a picture so as to use the display device 306 to view the probed position inside the human body or transmit the image to a computer.

When the image type intubation-aiding device 30 is in use, the light emission devices 3030 with several wavelength bands in the housing 3022 emit light. The light is transmitted through the transparent housing 3022 and reflected by a target. Making use of the light emission devices 3030 with several wavelength bands to probe the human body can detect out the variation of disease region to produce special images. After illumination by the light source module 3026 integrated with the housing 3022 and light collection by the light-collecting lens 3024 to produce an optical signal, which is focused by the focusing lens 3034 in the lens holder 3042. The optical signal is converted into an electric signal by the image sensor 3038 and then displayed on the display device 306 after processing. A common AC power, a battery, or a rechargeable battery provides the electric power for operation.

In the optical and imaging device 3028, a CMOS image sensor is installed behind the light emission devices 3030. Light reflected by the human body is focused by an object lens onto the CMOS image sensor, which converts the optical signal into an electric signal. The electric signal is processed by the image sensor drive circuit board 3036 and is then sent to the display device 306 via electric wires for real-time monitoring of images of the human body tissue. Further image processing can identify organs or nidus. Due to continual decrease of the feature size below 0.35 μm of the semiconductor fabrication process, the size of the CMOS image sensor will shrink constantly. Moreover, because of the packaging way changing from chip on board (COB) to chip size package (CSP), the packaged CMOS image sensor will be only slightly larger than the die. Besides, the size of the whole optical and imaging device 3028 can be reduced to be smaller than 5 mm due to progress of the fabrication technology of micro lens for the focusing lens 3034. The size of LED light source is also very small. It is hopeful that the outer diameter of the part penetrating into the human body of the whole device be smaller than 5 mm.

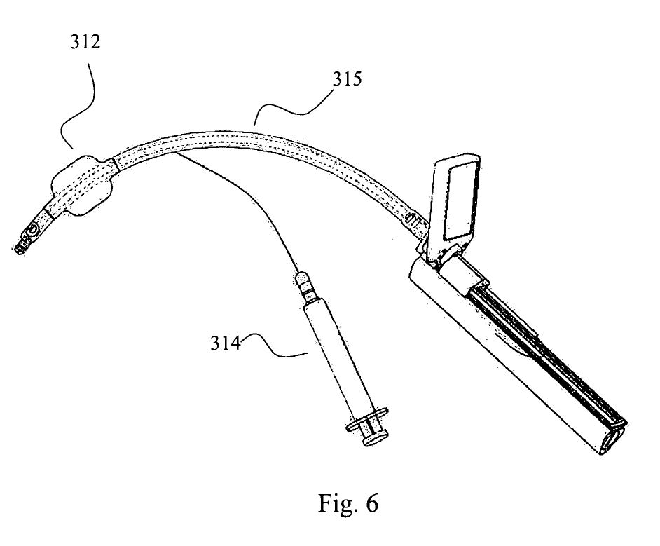

As shown in FIG. 6, the flexible soft tube 304 is placed in an endotracheal tube, an inflation bag 312 is installed in front of the endotracheal tube 315, and the inflation bag 312 is connected with an injector 314 for inflation. When an operator sticks the flexible soft tube 304 into the throat of a patient, he can inflate the inflation bag 312 using the injector 314. The endotracheal tube 315 can thus be fixed on the trachea of the patient to facilitate operation for medical staffs.

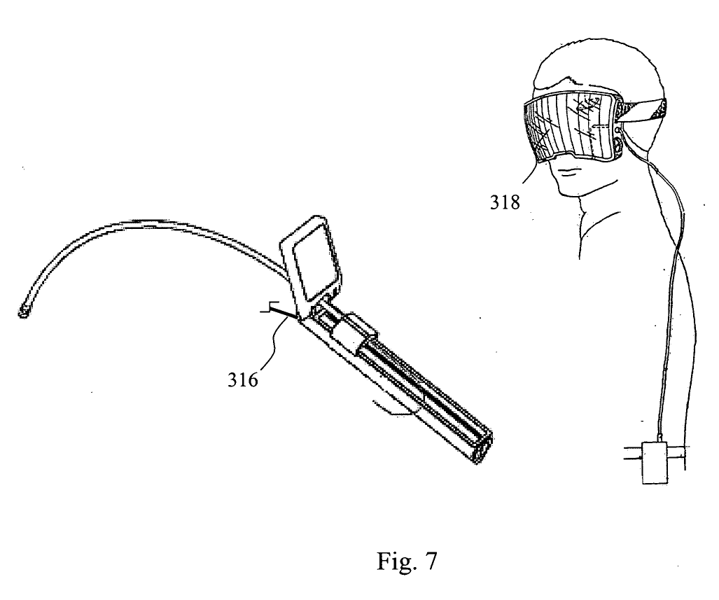

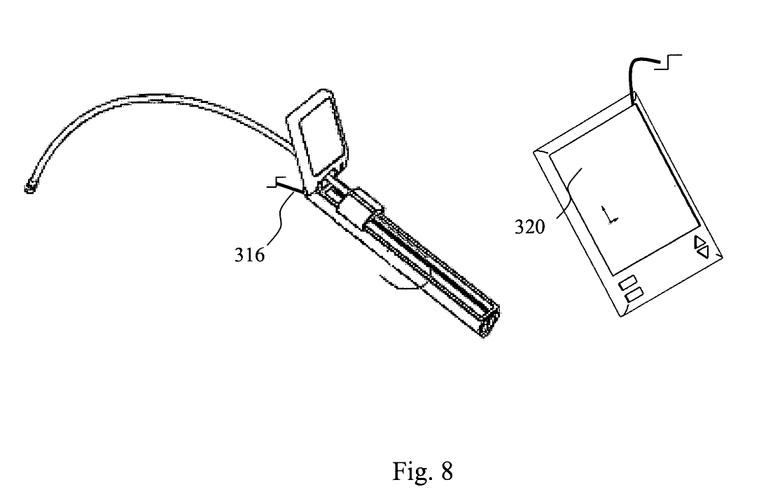

As shown in FIG. 7, a wireless transmission device 316 can be installed in the original image type intubation-aiding device to wirelessly transmit images to a mask type head-up display 318 or a handheld display 320 shown in FIG. 8. This function can facilitate use for medical staffs, and can also avoid infection of the medical staffs due to short-distance contact with the patient.

To sum up, the present invention provides an image type intubation-aiding device to help doctors with intubation of the human body. Through control of a handheld handle, the lens can be turned or moved to quickly find the position of trachea. Moreover, the advantages of the CMOS image sensor like small size and power saving and new optical techniques are made use of to increase the spot ratio of nidus. The conventional expensive and vulnerable fiber type endoscopes can be replaced to lower the cost. Moreover, disposable endoscopes are available to avoid infection of the human body due to repetitive use of endoscope.

Although the present invention has been described with reference to the preferred embodiments thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. An image-type intubation-aiding device comprising:
a probing device made of material compatible with the human body, said probing device comprising:
a housing;
a light source module disposed behind said housing for producing an optical signal for illuminating the front; and
an optical and imaging device disposed behind said light source module for converting said optical signal into an electric signal;
a flexible soft tube connected with said probing device;
a display device connected with said flexible soft tube and said optical and imaging device, said display device being used to receive said electric signal for displaying after processing; and
a power source device connected with all above said devices for providing electric power.

2. The image-type intubation-aiding device as claimed in claim 1, wherein the diameter of said housing is smaller than 15 mm.

3. The image-type intubation-aiding device as claimed in claim 1, wherein said housing is pervious to light.

4. The image-type intubation-aiding device as claimed in claim 1, wherein a light-collecting lens is disposed in said housing.

5. The image-type intubation-aiding device as claimed in claim 1, wherein said light source module comprises several light emission devices and a light source drive circuit for driving said light emission devices to emit light.

6. The image-type intubation-aiding device as claimed in claim 5, wherein said light emission devices are light-emitting diodes or organic light-emitting diodes.

7. The image-type intubation-aiding device as claimed in claim 1, wherein said optical and imaging device comprises a focusing lens and an image sensor.

8. The image-type intubation-aiding device as claimed in claim 7, wherein the visual angle of said focusing lens exceeds 360 degrees.

9. The image-type intubation-aiding device as claimed in claim 7, wherein said image sensor is a CMOS device or a CCD device for converting said optical signal into said electric signal.

10. The image-type intubation-aiding device as claimed in claim 1, wherein said display device is a liquid crystal display, an organic light emitting display, or a cathode-ray tube.

11. The image-type intubation-aiding device as claimed in claim 1, wherein said optical and imaging device is connected to said display device via at least an electric wire.

12. The image-type intubation-aiding device as claimed in claim 1, wherein said display device receives said electronic device in a wired or wireless way.

13. The image-type intubation-aiding device as claimed in claim 1, wherein said power source device is disposed in a handle connected with said display device.

14. The image-type intubation-aiding device as claimed in claim 13, wherein a control circuit is disposed in said handle for capturing a video or taking a picture.

15. The image-type intubation-aiding device as claimed in claim 13, wherein a thick metal wire is disposed in said flexible soft tube, and a soft tube retractable device connected with said thick metal wire and said handle is disposed behind said display device, and said handle can control said soft tube retractable device to push said thick metal wire for controlling the bend angle of said flexible soft tube.

16. The image-type intubation-aiding device as claimed in claim 1, wherein said electric signal is digital or analog.

17. The image-type intubation-aiding device as claimed in claim 1, wherein said power source device is a common AC power, a battery, or a rechargeable battery.

18. The image-type intubation-aiding device as claimed in claim 1, wherein several electric wires are disposed in said flexible soft tube for transmission of said electric power and said electric signal.

19. The image-type intubation-aiding device as claimed in claim 1, wherein a hole is formed on said flexible soft tube, and a biopsy device is disposed in said hole for sampling, sectioning, or inflation.

20. The image-type intubation-aiding device as claimed in claim 1, wherein said display device is rotatable.
